SEQUENCE LISTING (6 sequences)

SEQ ID NO: 1 - DNA, 20 bp, Artificial Sequence
  PCR PRIMER
cgaccatctgccgctttgag

SEQ ID NO: 2 - DNA, 20 bp, Artificial Sequence
  PCR PRIMER
ccccctgtcccccattccta

SEQ ID NO: 3 - DNA, 20 bp, Artificial Sequence
  PCR PRIMER
aaagaatcttcacctatgcc

SEQ ID NO: 4 - DNA, 20 bp, Artificial Sequence
  PCR PRIMER
gaaggaagaggagagacagt

SEQ ID NO: 5 - DNA, 24 bp, Artificial Sequence
  PCR PRIMER
cagatcctaaacagctcgcagaat

SEQ ID NO: 6 - DNA, 23 bp, Artificial Sequence
  PCR PRIMER
gcgtacgcaaattaaagtccaga

(19) United States
(12) Patent Application Publication
(10) Pub. No.: US 2013/0217120 A1
(43) Pub. Date: Aug. 22, 2013

(54) PLACENTA-DERIVED CELL-CONDITIONED CULTURE MEDIA AND ANIMAL-FREE, FEEDER-FREE METHOD FOR CULTURING STEM CELLS USING THE SAME

(71) Applicant: Korea University Research and Business Foundation, (US)

(72) Inventors: Byung Soo KIM, Seoul (KR); Seung-Jin LEE, Seoul (KR); Yong PARK, Seoul (KR); Ji Hye Hye JUNG, Seoul (KR); Ji Hye Hye KIM, Mungyong-si (KR)

(73) Assignee: Korea University Research and Business Foundation, Seoul (KR)

(21) Appl. No.: 13/858,493

(22) Filed: Apr. 8, 2013

Related U.S. Application Data
(62) Division of application No. 13/421,544, filed on Mar. 15, 2012.

(30) Foreign Application Priority Data
Apr. 18, 2011 (KR) 10-2011-0035470
Feb. 16, 2012 (KR) 10-2012-0016076

(51) Int. Cl. C12N 5/0735

(52) U.S. Cl. CPC C12N 5/0606
USPC 435/366; 435/405

(57) ABSTRACT

Disclosed are placenta-derived cell-conditioned culture media for stem cells. An animal-free, feeder-free method using the media is also provided for culturing stem cells. The media can prevent the stem cells from being contaminated with xenogeneic proteins or cells, and maintain human embryonic stem cells in an undifferentiated state for a long period of time in vitro with an economic benefit.

### CROSS-REFERENCE TO RELATED APPLICATIONS

This application is a divisional of U.S. patent application Ser. No. 13/421,544 (pending) filed on Mar. 15, 2012, which claims priority based on Korean Patent Application Nos. 10-2011-0035470 filed on Apr. 18, 2011 and 10-2012-0016076 filed on Feb. 16, 2012, the contents of which are incorporated herein by reference in their entirety.

### BACKGROUND OF THE INVENTION

1. Field of the Invention

The present invention relates to a placenta-derived cell-conditioned culture medium, and an animal-free, feeder-free culture method for stem cells using the same. More particularly, the present invention relates to a method for culturing human embryonic stem cells and induced pluripotent stem cells (hereinafter referred to as “iPS” or “iPSC”) using a human placenta-derived cell-conditioned medium by which neither animal sera nor feeder cells are necessary for the propagation of the human embryonic stem cells or iPSCs in undifferentiated states. Also, the present invention is concerned with the culture medium.

2. Description of the Related Art

Human embryonic stem cells are pluripotent cells, which are able to differentiate into all derivatives of the three primary germ layers (endoderm, ectoderm and mesoderm). Thus, they offer something to the development of medical treatments for a wide range of conditions, particularly hard-to-cure diseases including diabetes, liver cirrhosis, heart failure, cancer, etc. For use in the development of cell therapeutics, human embryonic stem cells should be cultured ex vivo safely for a long period of time while remaining undifferentiated. In 1998, Thomson first developed a technique to isolate and grow human embryonic stem cells in a cell culture (non-patent document 0001). Since then, advances have been made in applying human embryonic stem cells to cell therapeutics, but many problems with ex vivo culture techniques were disclosed. Some of them were overcome, but many of the basic problems remain unsolved.

According to the Thomson's technique of culturing human embryonic stem cells ex vivo, human embryonic stem cells are plated onto feeder cells (MEF, mouse embryonic fibroblast) in a medium supplemented with FBS (fetal bovine serum). Both FBS and MEF are respectively body fluid and cells of xeno-animal origin, but not human origin. Under the circumstance where they are cultured in contact with such xeno-impurities, human embryonic stem cells must be contaminated with animal proteins and cells. Cell therapeutics based on the human embryonic stem cells that are cultured under such conditions always have the possibility of introducing xeno-proteins and cells into patients, which may result in unexpected and serious adverse effects. Hence, a lot of research has been made into developing human embryonic stem cell culture systems free of xeno-impurities, such as animal proteins and cells.

As a replacement for FBS, a chemically defined, serum replacement (SR) was developed (non-patent document 0002) and is currently commercially available (20% Knockout Serum Replacement, KSR, Invitrogen). However, even though KSR is employed, the problem of xeno-contamination still remains because of the feeder cells of animal origin. Thus, the indispensability of feeder cells to maintaining human embryonic stem cells in an undifferentiated state pressed scientists to develop a technique of growing human embryonic stem cells in the presence of feeder cells of human origin. Among them are human foreskin fibroblast cells (HFF), human endothelial cells (HEC), human bone marrow mesenchymal stem cells (HMSC), and human placental cells (HPC). However, the use of feeder cells of human origin, although preventing contamination with xenogeneic proteins and cells, cannot avoid contamination with allogeneic proteins and cells unless the feeder cells are autologous to the embryonic stem cells. Because a human stem cell therapy product contaminated with xenogeneic proteins or cells may be rejected by the immune system of the patient, an ultimate requirement is a feeder-free culture system. Culture media for a feeder-free culture system originated from MEF-conditioned media (non-patent document 0003, 0004). Typically, MEF-conditioned media usable for human embryonic stem cells are prepared by adding elements essential for cell culturing to media, exposing the media to MEF for a certain time, and recovering the media. However, this feeder-free culture system using the MEF-conditioned media cannot be a true animal-free culture system. There always is the possibility of contamination with xenogeneic proteins and cells because the media is exposed to MEF. Recently, a medium completely free of xenogeneic proteins and cells has been developed and is commercially available (TeSR™2, STEMCELL TECHNOLOGIES).

Besides the medium, currently available feeder-free culture systems have another problem. Necessary for feeder-free culturing are contagious substances such as gelatin, instead of feeder cells, onto which stem cells are plated and attached. Human embryonic stem cells, in specific, require special gelatin-like substance, such as mouse cell-conditioned substance, for their culture, but typical gelatin cannot be used. It is understood that various cytokines and proteins useful for the survival and maintenance of human embryonic stem cells in undifferentiated states are absorbed into the gel during the conditioning process. The conditioned gel is currently commercially available (Matrigel®, BD Biosciences). Because Matrigel is produced by making contact with xenogeneic cells, there is always the risk of contamination with xenogeneic proteins and cells. Therefore, a culture medium completely free of xenogeneic proteins and cells (TeSR™2) does not guarantee a true animal-free culture system if Matrigel is used.

Conventional feeder-free culture systems also suffer from an economical disadvantage. The production of MEF-conditioned media or Matrigel is made possible by the sacrifice of many mouse adults or embryos, which costs a great deal. In addition, when human embryonic stem cells are cultured in MEF-conditioned media or on Matrigel, bFGF (basic fibroblast growth factor) must be continually added to the media or supporter to maintain the human embryonic stem cells in an undifferentiated state, which also costs a great deal. For use in the development of clinically applicable cell therapy products, human embryonic stem cells must be produced on a mass scale, but currently used feeder-free culture systems require high expenses for their operation and thus are regarded as economically unbeneficial.

Because isolating embryonic stem cells results in the death of the fertilized human embryo, the development of embryonic stem cell therapeutics raises ethical issues. Another barrier to the clinical use of embryonic stem cells is the immunological rejection that occurs when differentiated cells derived from embryonic stem cells are implanted into patients. Also, there is the problem of oncogenesis when incompletely differentiated cells are implanted. In an effort to overcome the above-mentioned problems, iPS (induced pluripotent stem) cell technology was developed to reprogram differentiated cells into pre-differentiated cells (Cell 132, 567-582, 2008). However, culturing iPS cells also suffers from the same problems as the mass production of embryonic stem cells.

As the background of the present invention, Korean Patent Laid-Open Publication No. 10-2007-0079586 (2007.08.07) discloses the development of human placenta stromal cells to promote human embryonic stem cells proliferation, differentiation of human embryonic stem cells to embryoid bodies and differentiation of human embryonic stem cells and tembryoid bodies to hemopoietic stem cells and Korean Patent Laid-Open Publication No. 10-2010-0006452(2010.01.19) discloses a method for maintenance of human embryonic stem cells using fibroblasts derived from human umbilical cord. In these prior techniques, the above-mentioned problems are present. That is, plating feeder cells is labor-intensive work so that it is economically unbeneficial while there is the possibility of contamination with xenogeneic proteins.

Therefore, there is a pressing need for a new culture method for the mass production of embryonic stem cells or embryonic stem cell-like cells that can avoid contamination with xenogeneic proteins or cells, such as those leading to immunological rejection, and is economically beneficial.

Many articles and patents are cited and quotations therefrom are provided throughout the specification. For a clear explanation of the background of the present invention and the content of the present invention, the disclosures of each of any such references in their entireties are hereby incorporated by reference into this application.

### SUMMARY OF THE INVENTION

Accordingly, the present invention has been made keeping in mind the above problems occurring in the prior art and the inventors have developed a method for culturing human embryonic stem cells in human placenta-derived feeder cell-conditioned media without Matrigel. The placenta is an organ that offers an environment essential for the survival of a developing fetus. In most cases, the placenta is discarded as medical waste after delivery. However, a great amount of scientific attention has been paid to the placenta since the discovery that cells derived from the chorion of the human placenta can be used as feeder cells for culturing human embryonic stem cells (non-patent document 0005). Further, it has recently been discovered that human placenta-derived feeder cells can produce bFGF, which is essential for the maintenance of human embryonic stem cells in an undifferentiated state, and thus can serve as feeder cells useful for maintaining the undifferentiated state of human embryonic stem cells without the supply of additional bFGF (non-patent document 0006). Taking notice of such properties of human placenta-derived cells, the present inventors exposed cytokine-free media to human placenta-derived cells (HPCs) for 24 hours (HPC conditioning) to prepare human placenta-derived cell-conditioned media (HPC-conditioned media) and succeeded in maintaining undifferentiated states of human embryonic stem cells or induced pluripotent stem cells for a long period of time in the HPC-condition media in gelatin-coated culture dishes.

It is therefore an object of the present invention to provide a placenta-derived cell (PC)-conditioned medium prepared through exposure to placenta-derived cells (PC conditioning) and a method for preparing the PC-conditioned medium.

It is another object of the present invention to provide an animal-free, feeder-free culture method for maintaining stem cells in the PC-conditioned medium whereby the stem cells can be completely prevented from direct or indirect contact with xenogeneic proteins and cells.

It is a further object of the present invention to provide a culture system that guarantees the mass production of embryonic stem cells in the absence of Matrigel or bFGF, with a higher economical benefit than in any other conventional serum-free, feeder-free culture systems.

The above objects may be accomplished by providing a method for preparing a placenta-derived cell-conditioned culture medium for stem cells, comprising: (a) seeding placenta-derived cells onto a gelatin-coated well plate; (b) adding a cell culture medium to the well plate and incubating the placenta-derived cells; and (c) recovering only the cell culture medium.

Also, the present invention provides a placenta-derived cell-conditioned culture medium for stem cells, prepared using the method.

Also, the present invention provides a method for culturing stem cells, comprising: adding the placenta-derived cell-conditioned culture medium for stem cells, prepared using the above method, to a gelatin-coated cell culture dish; and seeding stem cells into the cell culture dish.

### BRIEF DESCRIPTION OF THE DRAWINGS

The file of this patent contains at least one drawing executed in color. Copies of this patent with color drawings will be provided by the Patent and Trademark Office upon request and payment of the necessary fee.

The above and other objects, features and other advantages of the present invention will be more clearly understood from the following detailed description taken in conjunction with the accompanying drawings, in which:

FIG. 1 is of microphotographs showing the propagation of human placenta-derived cells. (A) 4 days after seeding into cell culture dishes: heterogeneous cell colonies are observed. (B) two weeks after seeding into cell culture dishes: homogeneous cell colonies of fibroblast morphology were observed. Magnification (A-B): X40

FIG. 2 is of photographs showing the effect of bFGF on the growth of the human embryonic stem cell line H1 in placenta-derived cell-conditioned media (PC-conditioned media; hereinafter interchangeably used with “PC-CM”) of the present invention. Even in the absence of bFGF, the human embryonic stem cell line H1 is maintained in an undifferentiated state in the PC-conditioned media (photographed on 10^{th} passage).

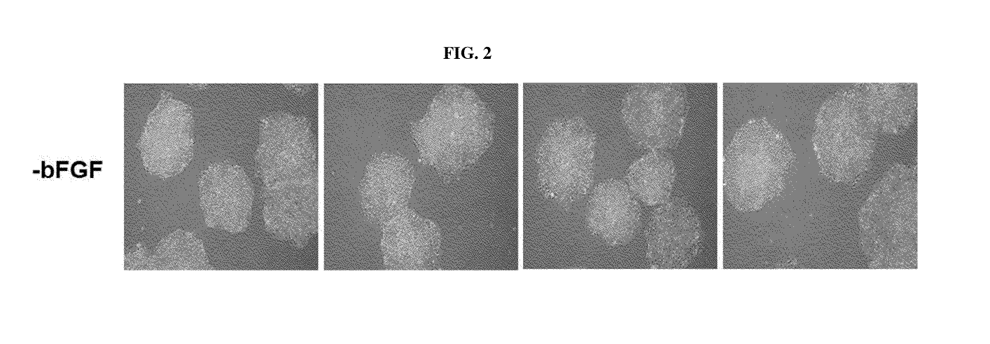

FIG. 3 is a photograph showing the expression of the sternness marker ALP on the human embryonic stem cell line H1 grown in the PC-conditioned media of the present invention (photographed on 10^{th} passage).

FIG. 4 is of photographs showing the expression of the typical markers of undifferentiated stem cells such as SSEA4, TRA60, TRA81 and OCT4 on the human embryonic stem cell line H1 grown in the PC-conditioned media of the present invention (photographed on 10^{th} passage).

FIG. 5 is a photograph showing the expression of the typical markers of undifferentiated stem cells such as Rex1, Nanog and Oct4 on the human embryonic stem cell line H1 grown in the PC-conditioned media of the present invention irrespective of the supplementation of bFGF, as analyzed by RT-PCR (on 10^{th} passage).

FIG. 6 is a graph showing the effect of the PC-conditioned media of the present invention on the maintenance of undifferentiated stem cells. The cell line H1 was cultured under the following four conditions: in PC-conditioned media of the present invention on a gelatin-coated culture dish without the supplementation of bFGF (PCCM-bFGF(G)), in the PC-conditioned media of the present invention on Matrigel without the supplementation of bFGF (PCCM-bFGF(M)), in the PC-condition media of the present invention on a gelatin-coated culture dish supplemented with bFGF (PCCM+bFGF(G)), and in the PC-conditioned media of the present invention on Matrigel supplemented with bFGF (PCCM+bFGF(M)). This experiment was repeated three times with n=4. Because of cell densities differing from one culture to another, stemness is expressed as percentages (%) of undifferentiated cell counts to total cell counts.

FIG. 7 is of photographs showing the human embryonic stem cells grown in the conditions of FIG. 6 (photographed on the 10^{th} passage at magnification x40). The degree of differentiation is apparently different for the groups supplemented with and without bFGF. The most undifferentiated state was detected when the cells were cultured in the media on the gelatin-coated culture dishes without the supplementation of bFGF, as measured by immunostaining against alkaline phosphatase.

FIG. 8 is of photographs of human induced pluripotent stem cells (iPSCs) grown in the PC-conditioned media on gelatin-coated culture dishes without the supplementation of bFGF (PC-CM-bFGF) (photographed on the 8^{th} passage at magnification ×40).

FIG. 9 is a photograph showing the expression of the typical markers of undifferentiated stem cells such as Rex1, Nanog and Oct4 on the human embryonic stem cell line H1 and iPSCs after 10 passages in PC-conditioned media on gelatin-coated culture dishes without the supplementation of bFGF.

FIG. 10 is of photographs showing the expression of the typical markers of undifferentiated stem cells such as Nanog, TRA-60, TRA-81, SSEA-4, and OCT-4 on the human embryonic stem cell line H1 and iPSCs grown in PC-conditioned media on gelatin-coated culture dishes without the supplementation of bFGF, as assayed by immunostaining (SSEA-1: negative control, DAPI: for live cell DNA staining, magnification ×40).

FIG. 11 is of photographs showing the pluripotency of the stem cells maintained in the PC-conditioned media on a gelatin-coated culture dish without the supplementation of bGFF (PC-CM-bFGF). After being grown in PC-CM-bFGF, the cell line was induced to form embryoid bodies for 2 weeks in vitro and allowed to grow for 10 days in an adherent pattern on a gelatin-coated culture dish. The cells were immunostained against AFP (endoderm), DESMIN (mesoderm) and TUJ1 (ectoderm) before observation under a fluorescence microscope at magnification ×200. Their ability to differentiate into the three layers was identified.

FIG. 12 is of photographs showing human iPSCs grown in the PC-conditioned media on a gelatin-coated culture dish without the supplementation of bGFF (PC-CM-bFGF) under the same conditions as in FIG. 11. Like the embryonic stem cell line, iPSCs were found to remain pluripotent.

FIG. 13 shows the pluripotentcy of the cells grown according to the method of the present invention, as analyzed by quantitative real-time PCR. RNA was extracted from the embryoid bodies formed after differentiation for two weeks, and used to synthesize complementary DNA. As a control, the human embryonic stem cell line which remained undifferentiated was used. The housekeeping gene GAPDH was used for the normalization of the cDNA. The typical markers of undifferentiated stem cells such as OCT-4 and NANOG were detected at a high level while the markers of the three germ layers endoderm, mesoderm and ectoderm were normally expressed in the groups which were subjected to induction for differentiation.

FIG. 14 shows the composition of the PC-conditioned medium of the present invention, as assayed by the cytokine antibody array kit (Raybiotech Inc., www.raybiotech.com). A total of 120 cytokines were detected. A basic medium that was not conditioned with human placenta-derived cells and the commercially available medium mTeSR were used as controls. The results of the experiment groups were normalized to those of the basic medium. The cytokines which had significantly different expression levels from those of mTeSR were selected.

FIG. 15 shows quantitative analysis results of the membrane array of FIG. 14 as measured in terms of density. The left panel is a table summarizing the cytokines having an expression level that was more than five-fold different between the groups after the value of each spot was numerized using the sicon program and averaged. The proteins are expressed in the pink rows when their expression levels are higher in the control and in the yellow rows when their expression levels are higher in the experimental groups. The right panel is a graph in which the expression levels are depicted, with top 5 of the highest expression levels being expressed using red.

FIG. 16 shows ENTREZE ID Nos. and protein names of the top 4 cytokines which have the greatest difference in expression level in FIG. 15.

FIG. 17 is of graphs showing the expression levels of the top 4 cytokines which have the greatest difference in expression level, as measured by enzyme linked immunosorbent assay (ELISA). As controls, a basic medium (Cont) which was not exposed to placenta-derived cells and the commercially available medium mTeSR(Con2) were used. The experiment was repeated three times with five randomly selected samples (unit=pg/ml).

FIG. 18 shows karyotypes of the stem cell lines grown in the PC-conditioned medium of the present invention for a long period of time. Both H1 and iPSC were found to retain the normal karyotype XY 46 as measured by G-bending analysis. Analysis was performed on the 17^{th} passage for H1 and on 10^{th} passage for iPSC.

### DESCRIPTION OF THE PREFERRED EMBODIMENTS

In accordance with an aspect thereof, the present invention pertains to a method for preparing a placenta-derived cell-conditioned culture medium for stem cells, comprising: (a) seeding placenta-derived cells onto gelatin-coated well plates; (b) adding a cell culture medium to the well plate and incubating the placenta-derived cells; and (c) recovering only the cell culture medium.

The placenta is usually expelled within tens of minutes after delivery. Immediately after being separated from the wall of the uterine, the placenta may be stored in ice-filled sterile vessel.

In one preferred embodiment, the method may further comprises separating the placenta-derived cells from the chorionic plate, culturing the placenta-derived cells in DMEM for passages, and treating the cells with trypsin and radiation prior to step (a).

In another embodiment of the method, the placenta-derived cells of step (a) may be placenta-derived fibroblast-like cells separated from the human chorionic plate.

In another embodiment of the method, the cell culture medium of step (b) may be a typical medium free of fetal bovine serum. Preferable is DMEM/F-12 supplemented with a serum replacement, an antioxidant and an antibiotic. For example, 10 ml of DMEM/F-12 supplemented with 20% knockout serum Replacer (GIBCO), 0.1 mM β-mercaptoethanol, and 1% penicillin-streptomycin (Sigma) may be used.

According to another embodiment, the placenta-derived cells may be preferably incubated for 20-30 hours in step (b) and more preferably for 24 hours.

As used herein, the term “stem cells” refers to cells that can replicate infinitely and differentiate into specialized cells under a suitable condition, and is intended to encompass adult stem cells, embryonic stem cells and embryonic stem cell-like cells. That is, so long as they exhibit pluripotency, multipotency or unipotency, any stem cells may be used in the present invention. Preferred are embryonic stem cells or induced pluripotent stem (iPS) cells.

Contemplated in accordance with another aspect of the present invention is a placenta-derived cell-conditioned culture medium for stem cells.

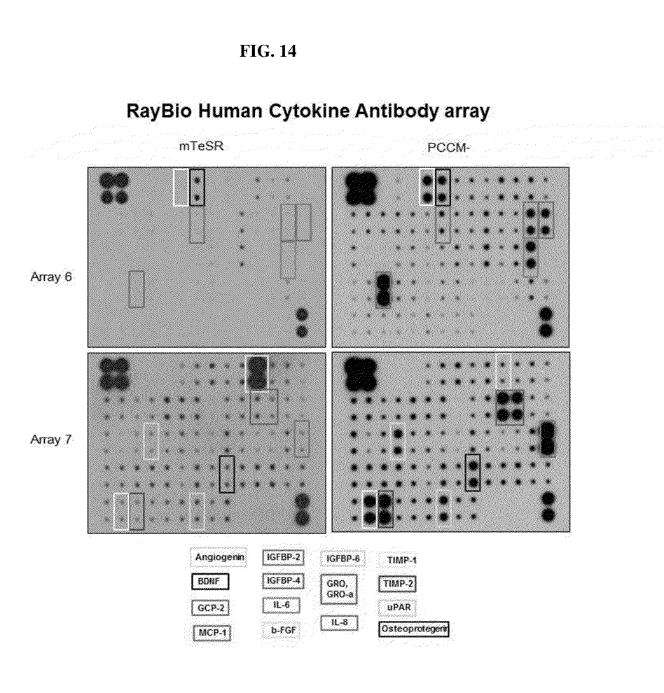

The placenta-derived cell-conditioned culture medium for stem cells contains factors secreted from placenta-derived cells, including bFGF essential for the maintenance of undifferentiated human embryonic stem cells and at least one cytokine selected from the group consisting of IL-8 (Atypical methylation of the interleukin-8 gene correlates strongly with the metastatic potential of breast carcinoma cells. Proc. Natl. Acad. Sci. U.S.A. 100: 13988-13993.), osteoprotegerin (Osteoprotegerin ligand is a cytokine that regulates osteoclast differentiation and activation. Cell 93, 165-176 (1998).), uPAR (uPAR: a versatile signalling orchestrator. Nat Rev Mol Cell Biol 3: 932-943 (2002)), TIM-1 (Identification of Tapr (an airway hyperreactivity regulatory locus) and the linked Tim gene family. Nat Immunol 2001; 2: 1109-.16.), TIM-2 (The TIM gene family: emerging roles in immunity and disease. Nat Rev Immunol 3: 454-462.), IGFBP-6 (Insulin-like growth factor binding protein-6 activates programmed cell death in non-small cell lung cancer cells. Oncogene 2000; 19: 4432-4436.), ICAM-1 (Structural plasticity in Ig superfamily domain 4 of ICAM-1 mediates cell surface dimerization. Proc Natl Acad Sci USA 104: 15358-15363.), angiogenin (ANG mutations segregate with familial and “sporadic” amyotrophic lateral sclerosis. Nat Genet. 2006, 38: 411-.413), BDNF (Cell Type-Specific Loss of BDNF Signaling Mimics Optogenetic Control of Cocaine Reward. Science 2010; 330-385), IGFBP-4 (Differential expression and biological effects of insulin-like growth factor-binding protein-4 and -5 in vascular smooth muscle cells. J. Biol. Chem. 273, 16836-.16842), IGFBP-2 (Insulin-like growth factor binding protein 2 is a growth inhibitory protein conserved in zebrafish. Proc Natl Acad Sci USA 96: 15274-15279.), IL-6 (Impaired immune and acute-phase responses in interleukin-6-deficient mice. Nature 368, 339-342.), GLP-2 (The proglucagon-derived peptide, glucagon-like peptide-2, is a neurotransmitter involved in the regulation of food intake. Nat Med, 6 (2000), pp. 802-807; Glucagon-like Peptide (GLP)-2 Reduces Chemotherapy-associated Mortality and Enhances cell Survival in Cells Expressing a Transfected GLP-2 Receptor Cancer Res 2001; 61:687-693.), MCP-1 (miR-124a as a key regulator of proliferation and MCP-1 secretion in synoviocytes from patients with rheumatoid arthritis. Ann Rheum Dis. 2011 March; 70 Suppl 1:i88-91.), GRO (Growth-regulated oncogene is pivotal in thrombin-induced angiogenesis. Cancer Res. 2006 Apr. 15; 66(8):4125-32.), and GRO-α (GRO α regulates human embryonic stem cell self-renewal or adoption of a neuronal fate. Differentiation 81 (2011) 222-232) (see FIGS. 14˜17) (Derivation of human embryonic stem cells in defined conditions. Nat. Biotechnol. 2006 February; 24(2):160-1.)

In one preferred embodiment, the placenta-derived cell-conditioned culture medium for stem cells in accordance with the present invention contains at least one cytokine selected from the group consisting of IL-8, MCP-1, TIMP-2, and GRO-α.

In accordance with a further aspect thereof, the present invention pertains to a method for culturing stem cells, comprising: adding a placenta-derived cell-conditioned culture medium for stem cells to a gelatin-coated cell culture dish; and seeding stem cells into the cell culture dish.

The stem cell-culturing method of the present invention may be applied to stem cells of various animals including dogs, cows, sheep, etc., as well as humans. Preferably, the placenta is of human origin, and the stem cells are embryonic stem cells or induced pluripotent stem (iPS) cells. More preferably, the embryonic stem cells are of human origin.

Because they take advantage of mouse-derived cells (MEF), conventional conditioned cell culture media have a high potency to contaminate human stem cells with xenogeneic proteins and cells. The cell culture system according to the present invention employs HPC-conditioned media as established by the present inventors for the first time in the art. Thus, the culture system can improve the clinical utility of human embryonic stem cells or iPS cells because it fundamentally excludes contamination by xenogeneic proteins or cells.

Unlike conventional feeder-free culture systems, the placenta-derived cell-conditioned media according to the present invention does not require coating the cell culture dish with Matrigel because they retain various cytokines and proteins necessary for the maintenance and survival of undifferentiated stem cells. Hence, the culture system of the present invention needs a gelatin coating, but not expensive Matrigel, for culturing human embryonic stem cells, and is economically advantageous.

Further, the steady supply of bFGF is requisite for the maintenance of human embryonic stem cells in conventional feeder-free culture systems, costing a great deal. In contrast, the conditioned media of the present invention does not need the additional supply of bFGF because they contain bFGF that is secreted by the placenta-derived cells.

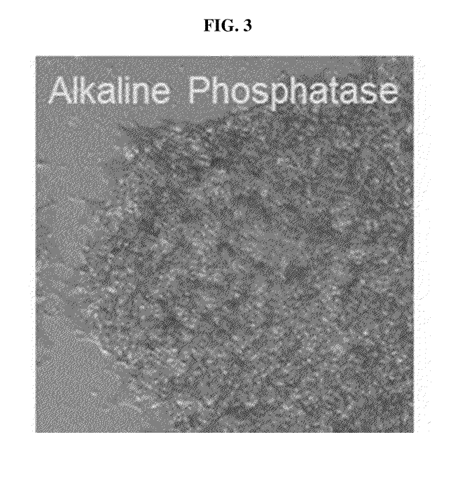

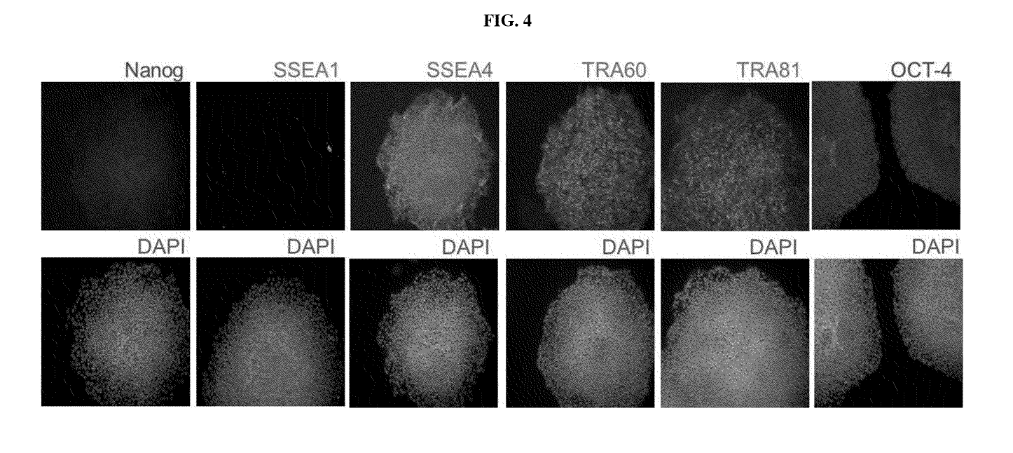

As shown in FIGS. 3 to 10, the undifferentiation of stem cells cultured in the animal-free, feeder-free, placenta-derived cell-conditioned media can be monitored by alkaline phosphatase (ALP) assay. In addition, the stem cells cultured according to the method of the present invention were observed to express cell surface markers typical of undifferentiated cells, including SSEA4, TRA-40 and TRA-81. Moreover, the sternness markers Rex1, Nanog, and Oct4 were found in the embryonic stem cells cultured according to the present invention irrespective of the supplementation of bFGF.

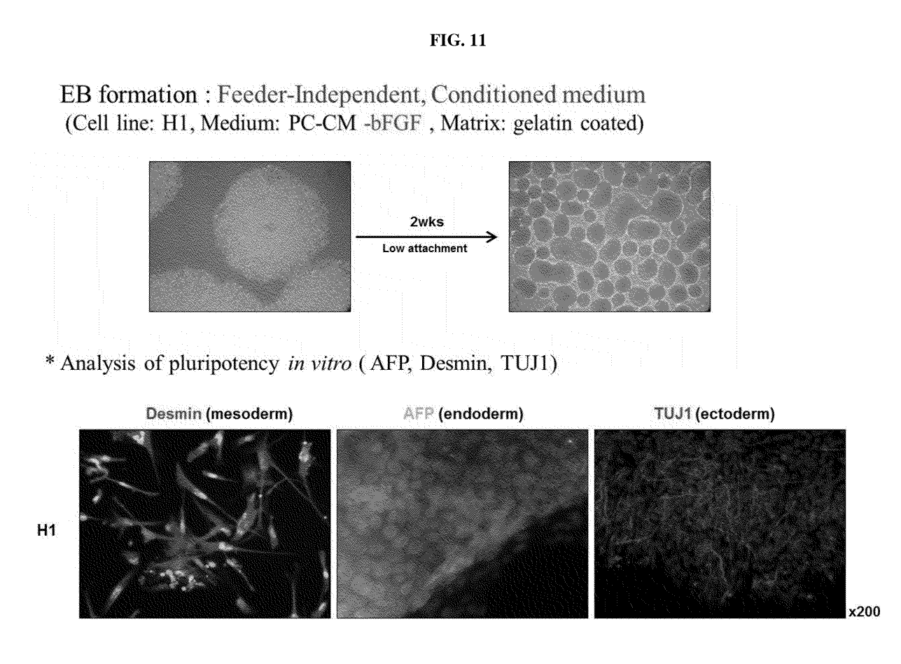

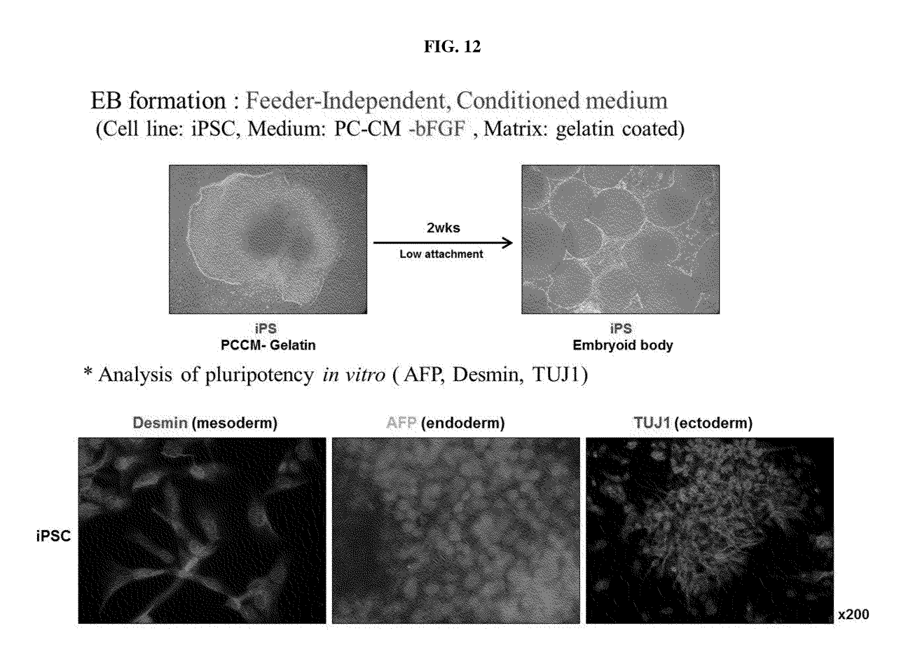

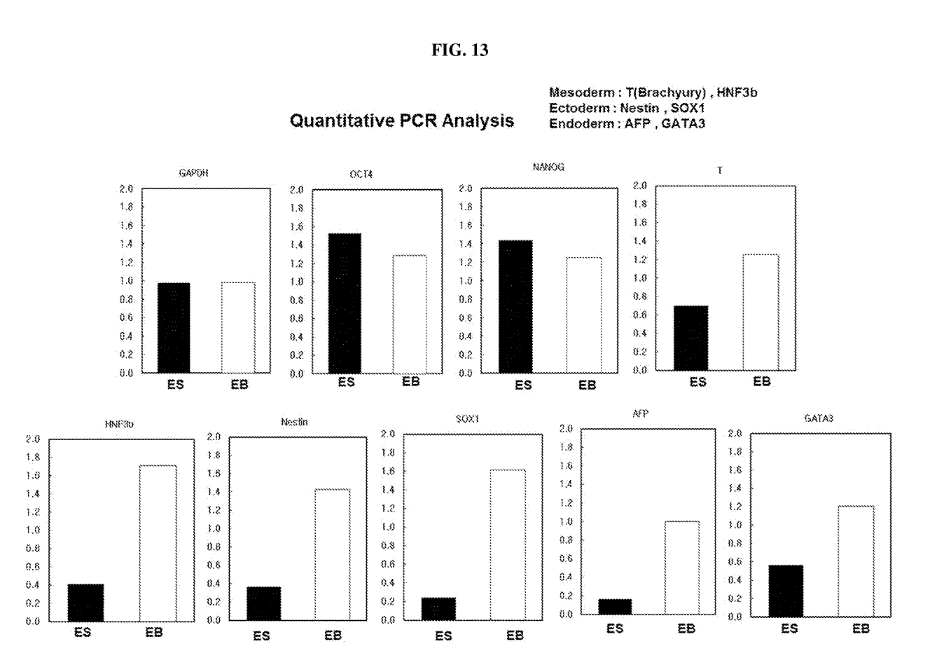

Embryonic stem cells are able to differentiate into all derivatives of the three primary germ layers (endoderm, ectoderm and mesoderm), in vivo or in vitro. When subjected to differentiation induction, the stem cells maintained in the placenta-derived cell-conditions culture media on gelatin in accordance with the present invention were found to differentiate into the three germ layers in vitro as measured by an immunostaining assay for markers of each germ layer (FIGS. 11 and 12). In addition, the differentiation potential of the stem cells cultured according to the method of the present invention was confirmed by quantitative real-time PCR analysis (FIG. 13).

Therefore, stem cells can be consistently cultured in an undifferentiated state for a long period of time using the method of the present invention.

In accordance with still a further aspect thereof, the present invention pertains to a placenta-derived cell-conditioned culture medium composition for stem cells, comprising at least one cytokine selected from the group consisting of bFGF, IL-8, osteoprotegerin, uPAR, TIM-1, TIM-2, IGFBP-6, ICAM-1, angiogenin, BDNF, IGFBP-4, IGFBP-2, IL-6, GLP-2, MCP-1, GRO, and GRO-α. In one preferred embodiment, the placenta-derived cell-conditioned culture medium composition for stem cells comprises at least one cytokine selected from the group consisting of IL-8, MCP-1, TIMP-2, and GRO-α.

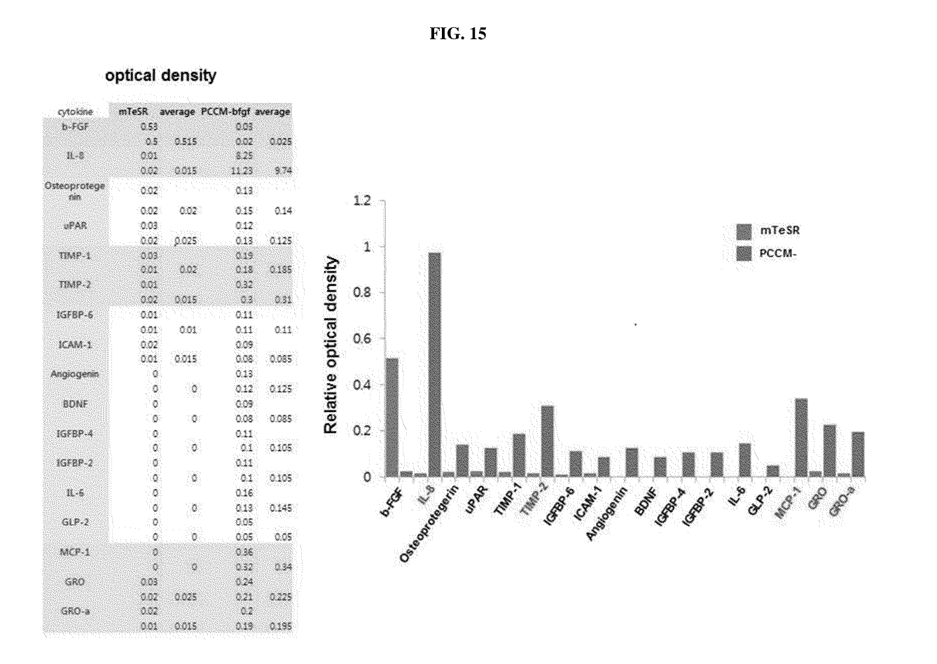

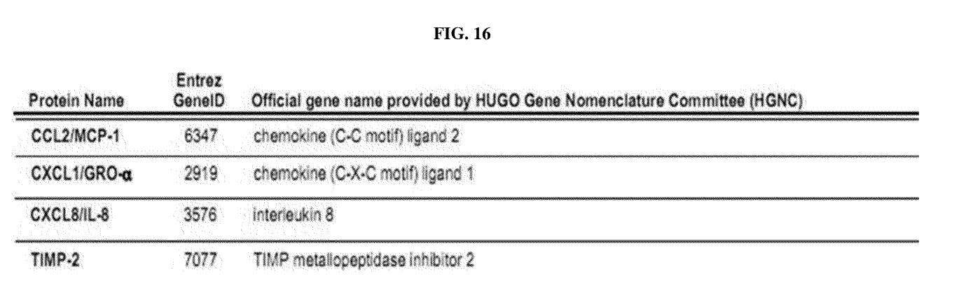

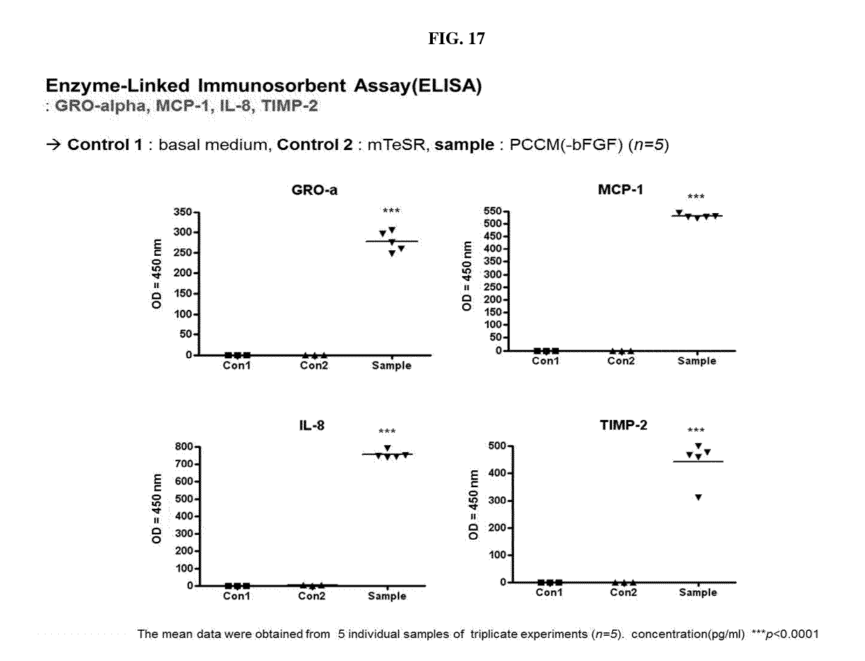

A total of 120 cytokines were identified as being contained in the placenta-derived cell-conditioned culture medium for stem cells of the present invention, as assayed by the human cytokine array, RayBiotech Inc. (FIG. 14). Comparison with mTeSR, commercially available from STEM CELL TECHNOLOGIES, showed that the expression levels of 17 cytokines in the placenta-derived cell-conditioned culture medium of the present invention are five or more times higher than those in mTeSR. A high level of bFGF, essential for the maintenance of undifferentiated human embryonic stem cells, is present in the commercially available culture medium mTeSR whereas the placenta-derived cell-conditioned culture media for stem cells contains relatively low levels of bFGF. Nonetheless, embryonic stem cells were observed to be maintained in an undifferentiated state for a long period of time in the placenta-derived cell-conditioned culture media of the present invention (FIG. 15). The top 4 cytokines with the greatest difference in expression level between the placenta-derived cell-conditioned media and mTeSR are IL-8, MCP-1, TIMP-2, and GRO-α. Their exact levels were determined by enzyme linked immunosorbent assay (ELISA) (FIGS. 16 and 17).

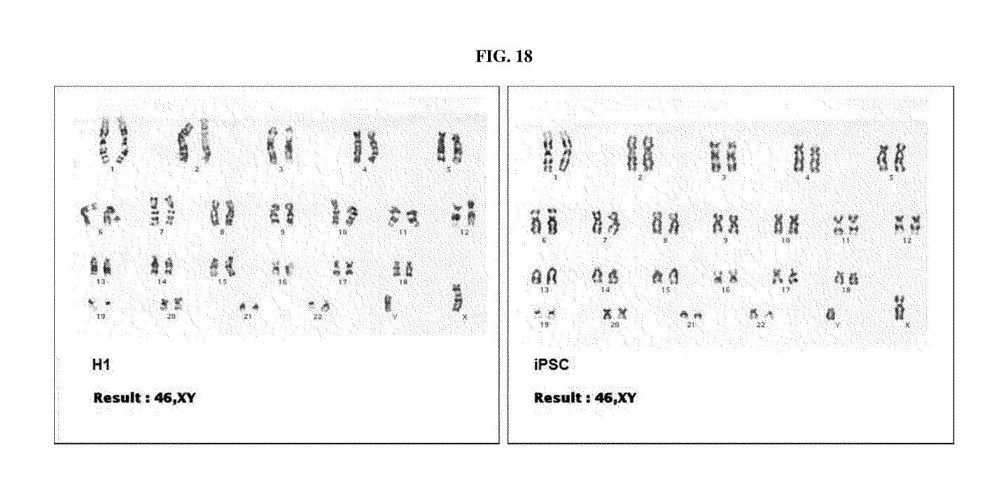

In addition, the stem cells cultured in the placenta-derived cell-conditioned medium of the present were identified as retaining a normal karyotype even after long passages (H1: p17, iPSC: p10) (FIG. 18).

A better understanding of the present invention may be obtained through the following examples which are set forth to illustrate, but are not to be construed as limiting the present invention.

### Example 1

### Production of Human Placenta-Derived Cells

Chorionic plates were excised from the placentae of healthy women by operation, minced and incubated at 37° C. for 30 min in 0.25% trypsin-EDTA (GIBCO-Invitrogen, Carlsbad, Calif.). Then, the cells were cultured at 37° C. and an RH of 95% in DMEM (Dulbecco's modified Eagle medium) supplemented with 20% fetal bovine serum (FBS; HyClone Laboratories Inc, Logan, Utah), 100 U/ml penicillin and 100 μg/ml streptomycin in a 5% CO₂ atmosphere.

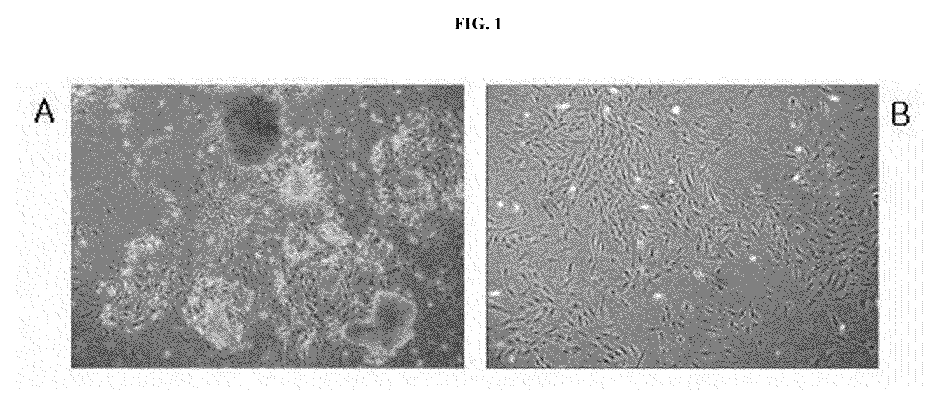

Until three passages, the medium was freshly exchanged every three to four days. During passages, floating cell debris was removed from the media. Placenta-derived fibroblast-like cells adherent to the well plates were visualized. From two weeks after the cells were seeded, placenta-derived fibroblast-like cells adherent to the well plates formed colonies (FIG. 1).

After being cultured until the 12^{th} passage, all the human placenta-derived cells (HPCs) were stocked. Before cryopreservation, the cells stock was trypsinized, treated with radiation (1500 cGy) and placed at a density of 1×10⁶ cells per cryovial. Also, RT-PCR was performed to examine whether the placentae were infected with typical pathogens among which are cytomegalovirus, herpes simplex virus types 1 and 2, Chlamydia trachomatis, Chlamydia spp, Mycoplasma genitalium, Mycoplasma hominis, and Ureaplasma ureaticum. In this context, well-know primer sequences were used (non-patent document 0007, 0008). HPCs which were positive to pathogens were discarded.

### Example 2

### Production of Placenta-Derived Cell-Conditioned (PC-Conditioned) Media

A frozen stock of HPCs was thawed and seeded at a density of 1×10⁶ cells/well into 35-mm well plates coated with 0.1% gelatin. To each well of the well plates was added 10 ml of DMEM/F-12 supplemented with 20% knockout serum replacer (GIBCO), 0.1 mM β-mercaptoethanol, and 1% penicillin-streptomycin (Sigma), followed by incubation for 24 hours at 37° C. and an RH of 95% in a 5% CO₂ atmosphere. Only the medium was recovered and stored at 4° C.

### Example 3

### In Vitro Maintenance and Propagation of Undifferentiated Human Embryonic Stem Cells in PC-Conditioned Media for Long Period of Time

The H1 human embryonic stem cell line was seeded into a gelatin cell culture dish containing an HPC-conditioned medium and cultured at 37° C. and an RH of 95% in a 5% CO₂ atmosphere, with the HPC-conditioned medium freshly changed every 48 hours. The H1 human embryonic stem cell line was passaged every week in a gelatin-coated cell culture dish and propagated in an undifferentiated state until at least the 20^{th} passage.

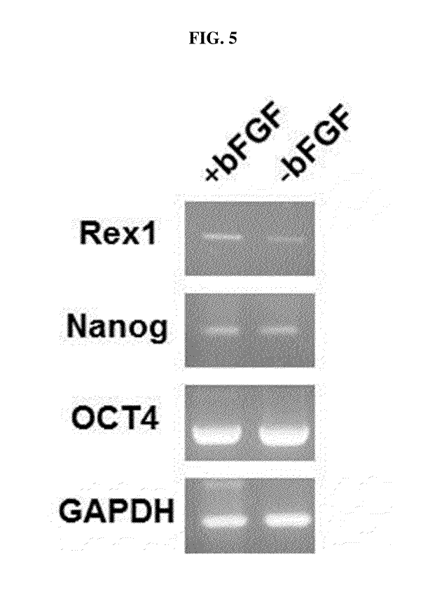

The undifferentiated state was confirmed optically every day by observation with an inverted microscope and biochemically every five passages by examining stemness markers through immunostaining against alkaline phosphatase (ALP), Oct-4, stage specific embryonic antigen (SSEA)-4, tumor rejection antigen (TRA)-60, and TRA-81 (FIGS. 3, 4, 7 and 10) and RT-PCR for Oct-4, Nanog, and Rex-1 (FIG. 5).

Primers used in the RT-PCR are as follows;

Oct-4, 577 bp:
(forward, SEQ ID NO: 1)
CGACCATCTGCCGCTTTGAG
(reverse, SEQ ID NO: 2)
CCCCCTGTCCCCCATTCCTA,
Nanog, 149 bp:
(forward, SEQ ID NO: 3)
AAAGAATCTTCACCTATGCC,
(reverse, SEQ ID NO: 4)
GAAGGAAGAGGAGAGACAGT,
Rex-1, 306 bp:
(forward, SEQ ID NO: 5)
CAGATCCTAAACAGCTCGCAGAAT,
(reverse, SEQ ID NO: 6)
GCGTACGCAAATTAAAGTCCAGA.

Concurrent cultures used for the immunocytochemistry were established in 6-well plates. Before analysis, the adherent cell layers were fixed at room temperature with 10% formalin (15 min), treated for 10 min with 0.1% Triton X-100/PBS, and incubated overnight at 4° C. with a primary antibody. A primary antibody against SSEA-4 was purchased from Hybridoma Bank (Hybricoma Bank, IA) and the other antibodies were purchased from Chemicon (Chemicon, CA). ALP activity was detected with a commercially available kit (Sigma). Some sternness markers were analyzed by RT-PCR. Total RNA was isolated using the QIAGEN RNeasy kit (Qiagen-Hilden, Germany). Reverse transcription was performed on 500 ng of the total RNA primed with random hexamers in the presence of AMV reverse transcriptase (Roche Molecular Biochemicals, Germany). PCR products thus obtained were separated on 1.5% agarose gel and visualized with ethium bromide.

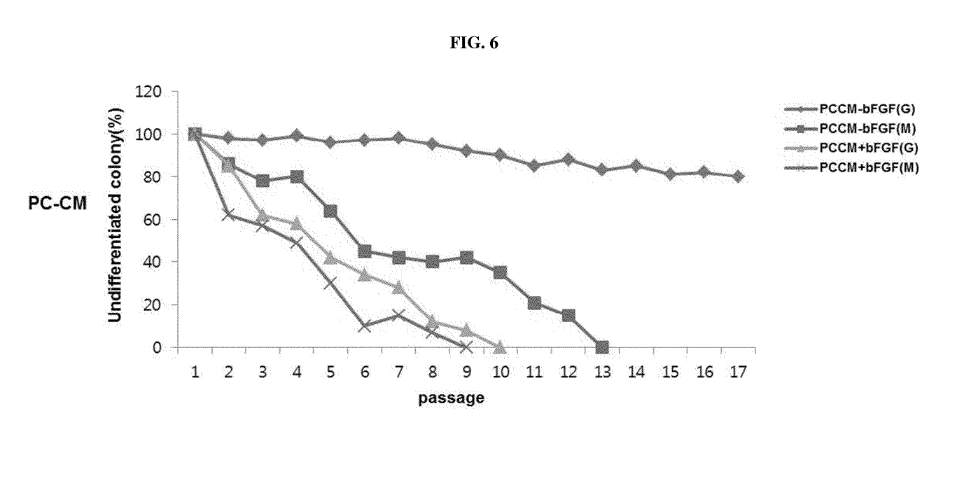

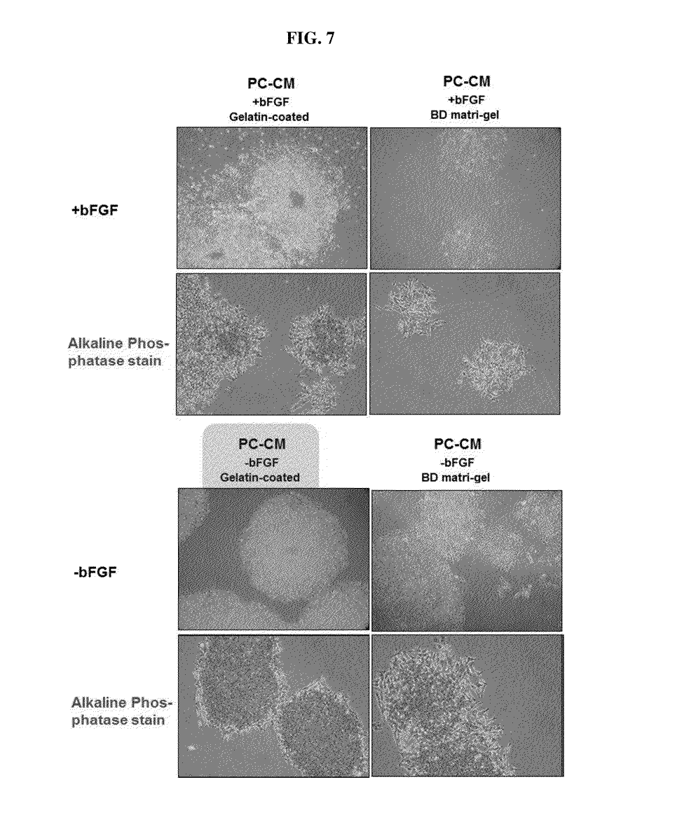

Further, a comparison was made between the PC-conditioned culture medium for stem cells of the present invention and the commercially available medium Matrigel. The human embryonic stem cell line H1 obtained from the WiCell Research Institute was used. The differentiation of the stem cells was determined by measuring the activity of alkaline phosphatase (ALP) (FIG. 7). The group grown in placenta-derived cell-conditioned cell culture medium on a gelatin-coated plate (PCCM-bFGF(G)) in accordance with the present invention was found to remain very undifferentiated until 17 passages (4-5 months) (FIGS. 6 and 7).

### Example 4

### Propagation of Undifferentiated Induced Pluripotent Stem Cell (iPS) in PC-Conditioned Media

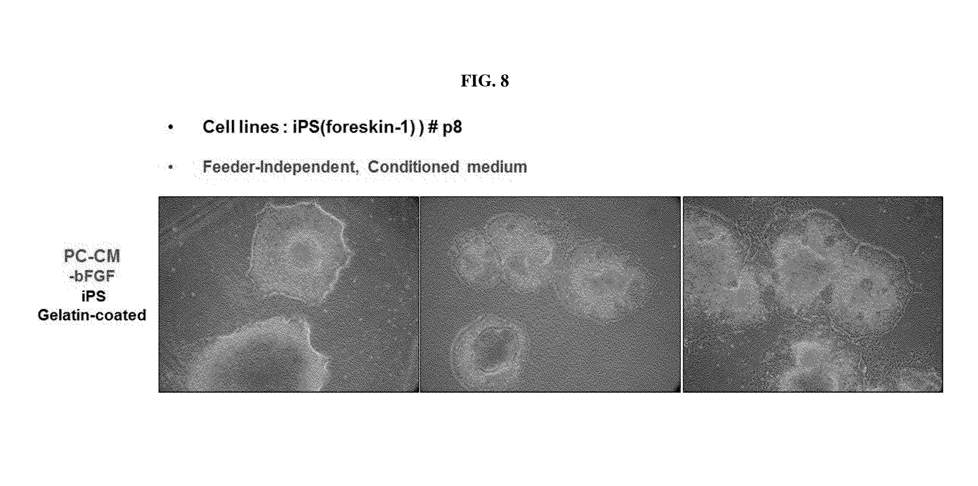

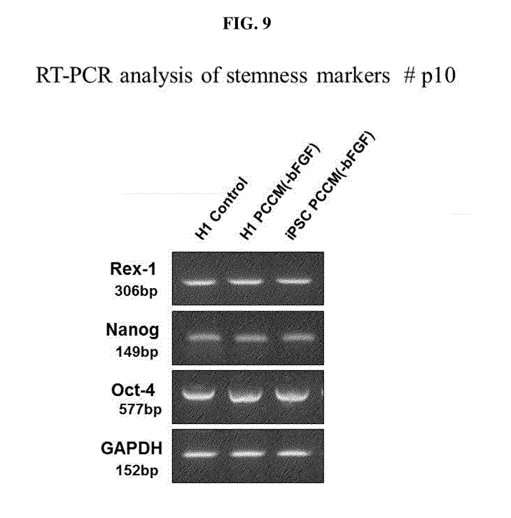

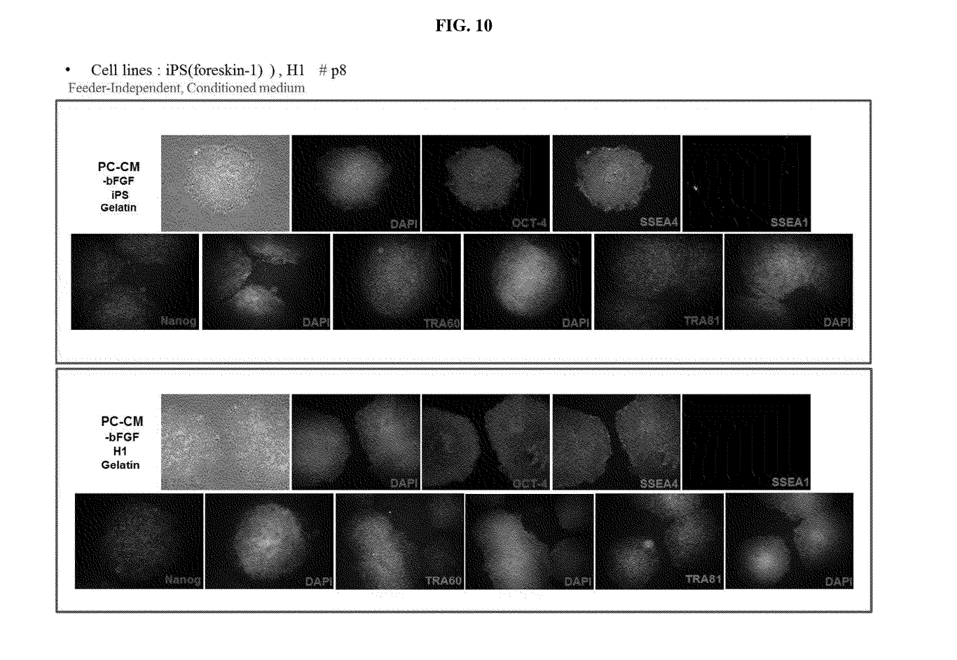

The PC-conditioned media of the present invention was applied to the iPS cell line (Foreskin-1) and the human embryonic stem cell line H1 and, both obtained from the WiCell Research Institute. Like the human embryonic stem cell line H1, iPS cells remained undifferentiated after many passages (FIG. 8). The stemness was also evaluated by RT-PCR for the markers Rex-1, Nanog and oct-4 (FIG. 9) and by immunostaining against the markers SSEA4, TRA-60, TRA-81, Nanog and oct-4 (FIG. 10).

### Example 5

### In Vitro Differentiation of Undifferentiated Human Embryonic Stem Cells and iPS Cells Grown in PC-Conditioned Media

Human embryonic stem cells are able to differentiate into all derivatives of the three primary germ layers (endoderm, ectoderm and mesoderm). Whether the stem cells grown in the HPC-conditioned media on gelatin-coated plates can be induced to differentiate into the three primary layers in vitro was evaluated as follows:

1) Immunohistochemical Staining

After being cultured for 14 passages according to the method of the present invention, undifferentiated human stem cells or iPSCs were enzymatically treated (dispase) to separate colonies which were then grown by suspension culture for two weeks in a culture condition for the formation of embryoid bodies so as to induce differentiation. The cells were then transferred to gelatin-coated dishes and cultured for 10 days in an adherent pattern. The medium for inducing differentiation contained 80% DMEM-F12, 20% knock-out serum, 0.1 mM β-mercaptoethanol, and 1% nonessential amino acids. The cytoplasm of the adherent cells was immunocytochemically stained to examine differentiation into endoderm, mesoderm and ectoderm. In this regard, the adherent cell layers were fixed at room temperature with 4% formalin (15 min), treated for 10 min with 0.1% Triton X-100/PBS, and incubated overnight with a primary antibody at 4° C. Primary antibodies against AFP and Desmin were purchased from SANTA CRUZ BIOTECHNOLOGY, Inc. and an anti-TUJ1 antibody from Chemicon (Chemicon, CA). The results are shown in FIGS. 11 and 12.

2) Quantitative PCR Analysis:

Total RNA was isolated from human embryonic stem cells, embryoid bodies and iPCs with the aid of High Pure RNA Isolation kit (Qiagen). From the total RNA, 2 μg of cDNA was synthesized in the presence of 200 units of reverse transcriptase using 500 ng of oligo(dT). For reverse transcription, the total RNA was incubated for 1 hour at 42° C. in a reaction mix containing 50 mM Tris-HCl (pH 8.3), 75 mM KCl, 3 mM MgCl₂, 10 mM dithiothreitol, and 1 mM dNTPs. Real-time PCR was performed in iCYCLER (BioRad) using 1×SYBR Green mix (Invitrogen), with 33 cycles of denaturation at 94° C. for 30 sec, annealing at 55° C. for 30 sec, and extension at 72° C. for 1 min. The results are shown in FIG. 13.

### Example 6

### Qualitative and Quantitative Analysis of PC-Conditioned Media and Discovery of Candidate Factor Essential for Maintenance of Undifferentiated Human Stem Cells

Compositions of the PC-conditioned cell culture media of the present invention and a commercially available culture medium for human embryonic stem cells were examined. A total of 120 cytokines were detected in the PC-conditioned cell culture media of the present invention as measured on a human cytokine array (RayBiotech Inc.). mTeSR (STEM CELL TECHNOLOGIES) was used as a control.

1) Human Cytokine Antibody Array:

The conditioned media of the present invention and the control were separately reacted with two sheets of nitrocellulose membrane on each of which 60 cytokines were printed (duplicate spots). After blocking, incubation, and washing steps, the membrane was incubated with biotin-conjugated antibodies and then with streptavidin-conjugated peroxidase and an ECL chemiluminescense reagent. Development was performed on autoradiographic films (BioMax Lite, Kodak). The experiment was repeated with five randomly selected samples, culminating in the detection of a total of 120 cytokines. The results of the experiment groups were normalized to those of the basic medium used as a control. The cytokines which had significantly different expression levels between the conditioned group and the mTeSR group were selected. The results are shown in FIGS. 14 to 16.

2) ELISA (Enzyme-Linked Immunosorbent Assay):

The levels of the candidate factors selected through the cytokine experiment were determined using Human ELISA kit (Raybiotech). The same experiment groups and control as in the previous experiment were used, and four cytokines were standardized. One hundred microliters of each of the samples from the experimental groups and the control group were reacted overnight at 4° C. and incubated for 1 hour with a biotin antibody. Subsequently, the samples were reacted with a streptavidin solution and a TMB One-Step substrate reagent, followed by termination with 50 μl of a stop solution. Absorbance at 450 nm was measured on a microplate reader. The experiment was conducted in triplicate for each cytokine. The results are shown in FIG. 17.

### Example 7

### Karyotype Analysis of Stem Cell Line Grown for Long Period of Time in PC-Conditioned Media

To evaluate chromosomal stability, karyotype analysis was performed of cultured hESCs and iPSCs each 17, 10 passages. ESCs were incubated with 0.1 μg/mL colcemid for 3-4 h, trypsinized, and then incubated in 0.075 M KCl for 20 min at 37° C. After fixation with 3:1 methanol/acetic acid, the karyotypes of ESCs were analyzed at 550-band resolution.

As described hitherto, the culture system for stem cells in accordance with the present invention is free of animal and feeder cells so that it is completely excluded from direct and indirect contact with xenogeneic proteins or cells. Hence, the animal-free, feeder-free cell culture system of the present invention does not incur the problem of xeno-contamination, and can guarantee the maintenance of human embryonic stem cells in an undifferentiated state for a long period of time in vitro.

In addition, the culture system of the present invention, based on the human placenta-derived cell-conditioned culture media, can maintain human embryonic stem cells with neither Matrigel nor the additional supply of bFGF, which is expensive, and thus have an economical advantage over conventional culture systems.

Consequently, the present invention is very useful for the cell therapy field using stem cells such as embryonic stem cells.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

### PRIOR ART DOCUMENT

### Patent Document

1. Korean Patent Laid-Open Publication No. 10-2007-0079586(2007.08.07)
2. Korean Patent Laid-Open Publication No. 10-2010-0006452(2010.01.19)

### Non-Patent Document

1. Thomson J A, Itskovitz-Eldor J, Shapiro S S, Waknitz M A, Swiergiel J J, Marshall V S, et al. Embryonic stem cell lines derived from human blastocysts. Science 1998 Nov. 6; 282(5391):1145-7.
2. Wiles M V, Johansson B M. Embryonic stem cell development in a chemically defined medium. Exp Cell Res 1999 Feb. 25; 247(1):241-8.
3. Xu C, Inokuma M S, Denham J, Golds K, Kundu P, Gold J D, et al. Feeder-free growth of undifferentiated human embryonic stem cells. Nat Biotechnol 2001 October; 19(10):971-4.
4. Roster E S, Fisk G J, Ares X, Irving J, Miura T, Rao M S, et al. Long-term culture of human embryonic stem cells in feeder-free conditions. Dev Dyn2004 February; 229(2):259-74.
5. Park Y, Choi I Y, Lee S J, Lee S R, Sung H J, Kim J H, et al. Undifferentiated propagation of the human embryonic stem cell lines, H1 and HSF6, on human placenta-derived feeder cells without basic fibroblast growth factor supplementation. Stem Cells Dev November; 19(11):1713-22.
6. Park Y, Kim J H, Lee S J, Choi I Y, Park S J, Lee S R, et al. Human Feeder Cells Can Support the Undifferentiated Growth of Human and Mouse Embryonic Stem Cells Using Their Own Basic Fibroblast Growth Factors. Stem Cells Dev February 25.
7. Genbacev O, Krtolica A, Zdravkovic T, Brunette E, Powell S, Nath A, et al. Serum-free derivation of human embryonic stem cell lines on human placental fibroblast feeders. Fertil Steril 2005 May; 83(5):1517-29.
8. McDonagh S, Maidji E, Ma W, Chang H T, Fisher S, Pereira L. Viral and bacterial pathogens at the maternal-fetal interface. J Infect Dis 2004 Aug. 15; 190(4):826-34.

What is claimed is:

1. A method for preparing a placenta-derived cell-conditioned culture medium, comprising:
(a) seeding placenta-derived cells onto a solid surface;
(b) bring a cell culture medium to be in contact with the placenta-derived cells and incubating the placenta-derived cells; and
(c) recovering only the cell culture medium from the culture obtained in (b) to obtain the placenta-derived cell-conditioned culture medium,
wherein said placenta-derived cell-conditioned culture medium is suitable for culturing stem cells.

2. The method of claim 1, wherein the placenta-derived cells of step (a) are placenta-derived fibroblast-like cells separated from a human chorionic plate.

3. The method of claim 1, wherein the basal cell culture medium of step (b) is DMEM/F-12 supplemented with a serum replacement.

4. The method of claim 1, wherein the incubation of step (b) is conducted for 20-30 hours.

5. The method of claim 1, wherein the solid surface in step (a) is a gelatin-coated surface.

6. The method of claim 1, wherein the placenta-derived cell-conditioned medium comprises b-FGF, IL-8, TIMP-2, MCP-1, GRO and GRO-α, and wherein the amount of b-FGF is smaller than the amount of each of IL-8, TIMP-2, MCP-1, GRO and GRO-α.

7. The method of claim 6, wherein the placenta-derived cell conditioned medium further comprises at least one cytokine selected from the group consisting of osteoprotegerin, uPAR, TIMP-1, IGFBP-6, ICAM-1, angiogenin, BDNF, IGFBP-4, IGFBP-2, IL-6, and GLP-2.

8. A method for culturing stem cells, comprising:
(a) providing the placenta-derived cell-conditioned culture medium of claim 1;
(b) seeding stem cells into the culture medium; and
(c) incubating the stem cells in the culture medium under conditions suitable for propagating the stem cells.

9. The method of claim 8, wherein the placenta is a human placenta and the stem cells are human embryonic stem cells.

10. The method of claim 8, wherein the placenta is a human placenta and the stem cells are induced pluripotent stem cells.

11. The method of claim 8, wherein the placenta-derived cell-conditioned medium comprises b-FGF, IL-8, TIMP-2, MCP-1, GRO and GRO-α, and wherein the amount of b-FGF is smaller than the amount of each of IL-8, TIMP-2, MCP-1, GRO and GRO-α.

12. The method of claim 11, wherein the placenta-derived cell conditioned medium further comprises at least one cytokine selected from the group consisting of osteoprotegerin, uPAR, TIMP-1, IGFBP-6, ICAM-1, angiogenin, BDNF, IGFBP-4, IGFBP-2, IL-6, and GLP-2.
